(19) United States
(12) Patent Application Publication
(10) Pub. No.: US 2024/0063462 A1
(43) Pub. Date: Feb. 22, 2024

(54) SYSTEMS AND METHODS FOR HEAT MANAGEMENT IN WIRELESS POWER TRANSFER SYSTEMS

(71) Applicant: TC1 LLC, St. Paul, MN (US)

(72) Inventors: Gionata VALCHERA, Zürich (CH); Adrian BOHL, Dietikon (CH); Simon RABOLD, Zürich (CH)

(21) Appl. No.: 18/268,276

(22) PCT Filed: Dec. 20, 2021
(86) PCT No.: PCT/US2021/064317

§ 371 Date: Jun. 19, 2023

Related U.S. Application Data
(60) Provisional application No. 63/128,513, filed on Dec. 21, 2020.

(51) Int. Cl. H01M 10/6551; H01M 10/0525; H01M 50/204; H01M 10/46; H01M 10/42; H01M 10/623; H01M 10/653; A61M 60/178; A61M 60/876

(52) U.S. Cl. CPC H01M 10/6551; H01M 10/0525; H01M 50/204; H01M 10/46; H01M 10/4257; H01M 10/623; H01M 10/653; A61M 60/178; A61M 60/876; H01M 2220/30; H02J 7/0042

(57) ABSTRACT

An implantable battery pack is provided. The implantable battery pack includes a housing, a plurality of battery cells positioned within the housing, and an electronics layout positioned within the housing, the electronics layout electrically coupled to the plurality of battery cells. The electronics layout includes at least one printed circuit board including electronics mounted thereon, and at least one thermally conductive wing coupled to and extending from the at least one printed circuit board, the at least one thermally conductive wing operable to spread heat generated by the electronics throughout the implantable battery pack.

### CROSS REFERENCE TO RELATED APPLICATIONS

This application claims priority to provisional application Ser. No. 63/128,513, filed Dec. 21, 2020, which is incorporated herein by reference in its entirety.

### BACKGROUND OF THE DISCLOSURE

### a. Field of the Disclosure

The present disclosure relates generally to wireless power transfer systems, and more specifically, relates to heat management in wireless power transfer systems.

### b. Background

Ventricular assist devices, known as VADs, are implantable blood pumps used for both short-term (i.e., days or months) and long-term (i.e., years or a lifetime) applications where a patient's heart is incapable of providing adequate circulation, commonly referred to as heart failure or congestive heart failure. A patient suffering from heart failure may use a VAD while awaiting a heart transplant or as a long term destination therapy. In another example, a patient may use a VAD while recovering from heart surgery. Thus, a VAD can supplement a weak heart (i.e., partial support) or can effectively replace the natural heart's function.

A wireless power transfer system may be used to supply power to the VAD. The wireless power transfer system generally includes an external transmit resonator and an implantable receive resonator configured to be implanted inside a patient's body. This power transfer system may be referred to as a transcutaneous energy transfer system (TETS).

In such systems, an implantable battery pack may be used to facilitate powering and controlling the VAD. The implantable battery pack may include, for example, lithium ion battery cells that can be charged relatively quickly at relatively high currents. However, lithium ion battery cells, as well as the electronics necessary to charge and discharge them, may also generate substantial amounts of heat. Accordingly, for implantable battery packs including lithium ion battery cells, it is important to effectively manage heat generated by those battery packs.

### SUMMARY OF THE DISCLOSURE

The present disclosure is directed to an implantable battery pack. The implantable battery pack includes a housing, a plurality of battery cells positioned within the housing, and an electronics layout positioned within the housing, the electronics layout electrically coupled to the plurality of battery cells. The electronics layout includes at least one printed circuit board including electronics mounted thereon, and at least one thermally conductive wing coupled to and extending from the at least one printed circuit board, the at least one thermally conductive wing operable to spread heat generated by the electronics throughout the implantable battery pack.

The present disclosure is further directed to an electronics layout for use in an implantable battery pack including a housing and a plurality of battery cells. The electronics layout includes at least one printed circuit board including electronics mounted thereon, and at least one thermally conductive wing coupled to and extending from the at least one printed circuit board, the at least one thermally conductive wing operable to spread heat generated by the electronics throughout the implantable battery pack.

The present disclosure is further directed to a method of assembling an implantable battery pack. The method includes positioning a plurality of battery cells within a housing, and electrically coupling an electronics layout to the plurality of battery cells. The electronics layout is positioned within the housing and includes at least one printed circuit board including electronics mounted thereon, and at least one thermally conductive wing coupled to and extending from the at least one printed circuit board, the at least one thermally conductive wing operable to spread heat generated by the electronics throughout the implantable battery pack.

### BRIEF DESCRIPTION OF THE DRAWINGS

FIG. 1 is a simplified electrical circuit diagram of one embodiment of a wireless power transfer system.

FIG. 2 is an illustration of the wireless power transfer system of FIG. 1 being used to supply power to a ventricular assist device (VAD).

FIG. 3A is atop view of one embodiment of an electronics layout that may be used with the implanted device shown in FIG. 2.

FIG. 3B is a bottom view of the electronics layout shown in FIG. 3A.

FIG. 4 is a perspective view of the electronics layout shown in FIGS. 3A and 3B in a folded configuration.

FIG. 5 is a schematic diagram illustrating heat flow through an implantable battery pack including the electronics layout shown in FIGS. 3A, 3B, and 4B.

FIG. 6 shows a thermal simulation for the implantable battery pack shown in FIG. 5.

FIG. 7 is a plan view of an alternative electronics layout.

FIG. 8 is an exploded view of an implantable battery pack including the electronics layout shown in FIG. 7.

FIG. 9 is a schematic diagram illustrating heat flow through the implantable battery pack shown in FIG. 8.

FIGS. 10 and 11 show thermal simulations for implantable battery packs.

### DETAILED DESCRIPTION OF THE DISCLOSURE

The present disclosure is directed to systems and methods for managing heat in wireless power transfer systems. An implantable battery pack includes a housing, a plurality of battery cells positioned within the housing, and an electronics layout positioned within the housing, the electronics layout electrically coupled to the plurality of battery cells. The electronics layout includes at least one printed circuit board including electronics mounted thereon, and at least one thermally conductive wing coupled to and extending from the at least one printed circuit board, the at least one thermally conductive wing operable to spread heat generated by the electronics throughout the implantable battery pack.

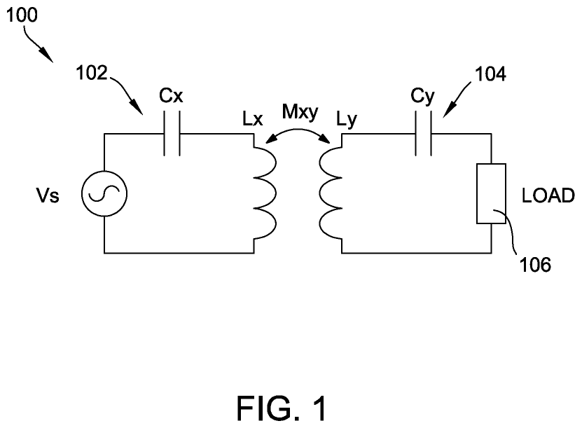

Referring now to the drawings, FIG. 1 is a simplified circuit of an exemplary wireless power transfer system 100. The system 100 includes an external transmit resonator 102 and an implantable receive resonator 104. In the system shown in FIG. 1, a power source Vs is electrically connected with the transmit resonator 102, providing power to the transmit resonator 102. The receive resonator 104 is connected to a load 106 (e.g., an implantable medical device). The receive resonator 104 and the load 106 may be electrically connected with a switching or rectifying device (not shown).

In the exemplary embodiment, the transmit resonator 102 includes a coil Lx connected to the power source Vs by a capacitor Cx. Further, the receive resonator 104 includes a coil Ly connected to the load 106 by a capacitor Cy. Inductors Lx and Ly are coupled by a coupling coefficient k. M_{xy} is the mutual inductance between the two coils. The mutual inductance, M_{xy}, is related to the coupling coefficient k as shown in the below Equation (1).

M_{xy}=k\sqrt{Lₓ·L_{y}} (1)

In operation, the transmit resonator 102 transmits wireless power received from the power source Vs. The receive resonator 104 receives the power wirelessly transmitted by the transmit resonator 102, and transmits the received power to the load 106.

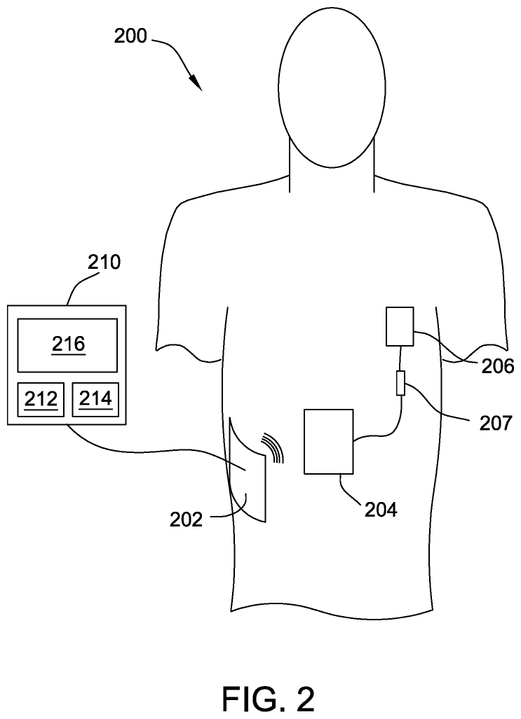

FIG. 2 illustrates one embodiment of a patient 200 using an external coil 202 (such as the transmit resonator 102 shown in FIG. 1) to wirelessly transmit power to an implanted coil 204 (such as the receive resonator shown in FIG. 1). The implanted coil 204 uses the received power to power an implanted device 206. For example, the implanted device 206 may include a pacemaker or heart pump (e.g., a left ventricular assist device (LVAD)). In some embodiments, the implanted coil 204 and/or the implanted device 206 may include or be coupled to a battery 207. For example, as shown in FIG. 2, battery 207 is coupled between implanted coil 204 and implanted device 206.

In one embodiment, the external coil 202 is communicatively coupled to a computing device 210, for example, via wired or wireless connection, such that the external coil 202 may receive signals from and transmit signals to the computing device 210. In some embodiments, the computing device 210 is a power source for the external coil 202. In other embodiments, the external coil 202 is coupled to an alternative power supply (not shown). The computing device 210 includes a processor 212 in communication with a memory 214. In some embodiments, executable instructions are stored in the memory 214.

The computing device 210 further includes a user interface (UI) 216. The UI 216 presents information to a user (e.g., the patient 200). For example, the UI 216 may include a display adapter (not shown) that may be coupled to a display device, such as a cathode ray tube (CRT), a liquid crystal display (LCD), an organic LED (OLED) display, and/or an “electronic ink” display. In some embodiments, the UI 216 includes one or more display devices. Further, in some embodiments, presentation interface may not generate visual content, but may be limited to generating audible and/or computer-generated spoken-word content. In the example embodiment, the UI 216 displays one or more representations designed to aid the patient 200 in placing the external coil 202 such that the coupling between the external coil 202 and the implanted coil 204 is optimal. In some embodiments, the computing device 210 may be a wearable device. For example, in one embodiment, the computing device 210 is a wrist watch, and the UI 216 is displayed on the wrist watch.

Implanted device 206 may be powered using an implanted battery pack including cells that are recharged by the power transfer between external coil 202 and implanted coil 204. As the implanted battery pack is an implanted device, it is important to manage thermal energy generated by the implanted battery pack. For example, to ensure proper operation, at least some regulations require that an outer surface of an implanted device have a temperature no greater than 2° C. above the temperature of surrounding tissue (i.e., 37° C.).

Implanted battery packs including lithium ion cells may generate additional thermal energy (relative to implanted battery packs including other types of cells). Accordingly, effective heat management solutions are needed to facilitate maintaining acceptable temperatures at an outer surface of such implanted battery packs. The more effective the heat management, the faster the lithium ion cells may be charged.

The systems and methods described herein facilitate spreading heat generated within an implanted battery pack substantially uniformly over an outer surface (e.g., a titanium housing) of the implanted battery pack. That is, the systems and methods described herein facilitate reducing hot spots on the outer surface.

As described herein, components that generate higher amounts of heat in the implanted battery pack (e.g., power conversion electronics) are located on a printed circuit board (PCB) proximate a center of the implanted battery pack. Further, the PCB is coupled to one or more thermally conductive wings to facilitate spreading heat throughout the battery pack. Graphite foils may also be positioned within the implanted battery pack to facilitate heat spreading.

Regarding heat dissipation, a sphere of a homogenous material with a heat source in the middle of the sphere would give a uniform thermal flux through the outer surface of the sphere. Obviously, an implanted battery pack has a different geometry and combinations of different materials. Accordingly, to facilitate relatively uniform heat dissipation in an implanted battery pack, thermally insulative and conductive materials are combined to conduct heat to particular locations on the outer surface of the battery pack, as described herein. Further, electronic components that contribute to generating and dissipating heat are arranged in particular locations within the battery pack. For example, in the embodiments described herein, power conversion circuitry is generally positioned in the middle of the battery pack.

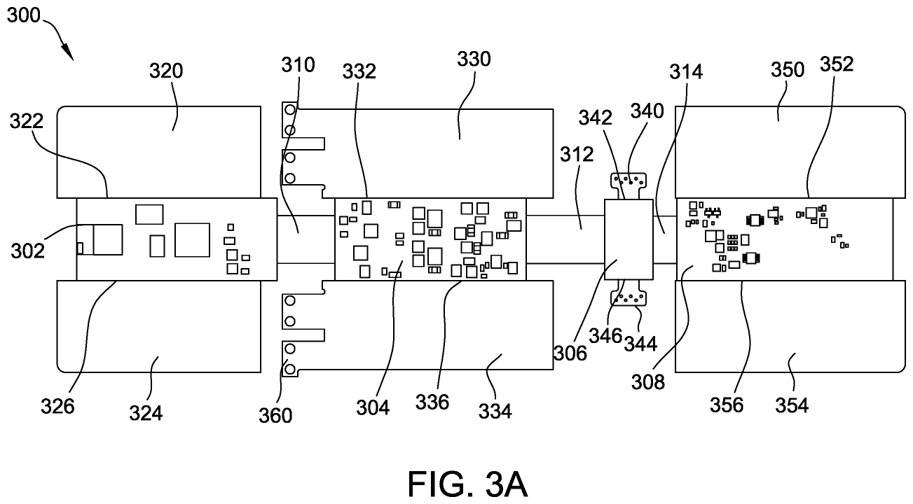

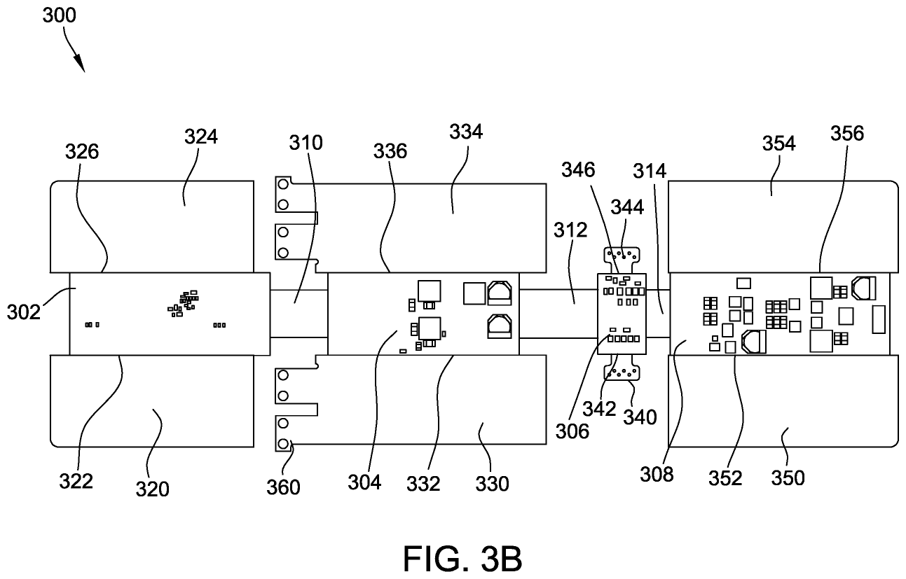

FIG. 3A is atop view of one embodiment of an electronics layout 300 that may be used with implanted device 206 (shown in FIG. 2). FIG. 3B is a bottom view of electronics layout 300. Electronics layout 300 is included in an implantable battery pack that facilitates powering implanted device 206. The implantable battery pack may be incorporated within implanted device 206 or may be coupled to implanted device 206.

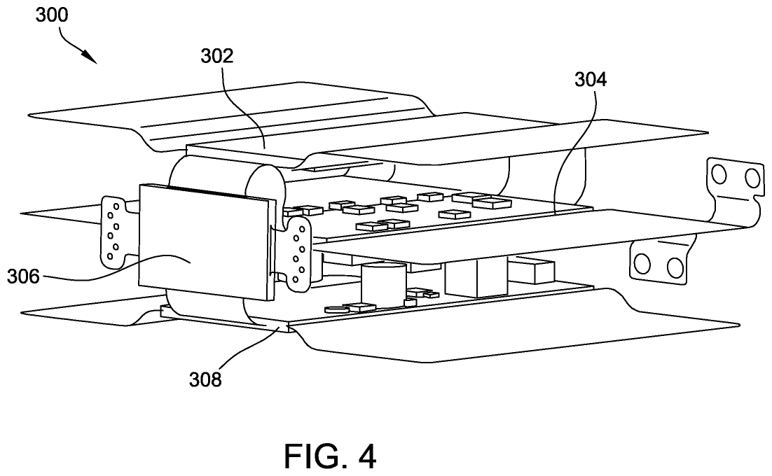

In FIGS. 3A and 3B, electronics layout 300 is shown in a flat, or unfolded configuration. As described herein, electronics layout 300 is positioned in a folded configuration (shown in FIG. 4) when located within the implantable battery pack. Electronics layout 300 facilitates evenly spreading heat generated by battery cells in the implantable battery pack, as described herein.

As shown in FIGS. 3A, 3B, and 4, electronics layout 300 includes a first PCB 302, a second PCB 304, a third PCB 306, and a fourth PCB 308. In this embodiment, a microcontroller and digital signal processing electronics are mounted to first PCB 302, power conversion electronics and battery charging electronics are mounted to second PCB 304, filtering electronics (e.g., electrostatic discharge electronics) are mounted to third PCB 306, and boost electronics (e.g., a DC-DC step up converter electronics) are mounted to fourth PCB 308. Alternatively, any suitable electronics may be mounted to PCBs 302, 304, 306, and 308.

Further, a first flexible thermally conductive connector 310 extends between first PCB 302 and second PCB 304, a second flexible thermally conductive connector 312 extends between second PCB 304 and third PCB 306, and a third flexible thermally conductive connector 314 extends between third PCB 306 and fourth PCB 308. Conductive connectors 310, 312, and 314 may be, for example, flexible PCBs that extend between relatively rigid PCBs 302, 304, 306, and 308.

To facilitate spreading heat, electronics layout 300 includes one or more thermally conductive wings coupled to relatively rigid PCBs 302, 304, 306, and 308. In the embodiment shown in FIGS. 3A, 3B, and 4, a first thermally conductive wing 320 is coupled to a first edge 322 of first PCB 302, and a second thermally conductive wing 324 is coupled to a second, opposite edge 326 of first PCB 302. Similarly, a third thermally conductive wing 330 is coupled to a first edge 332 of second PCB 304, and a fourth thermally conductive wing 334 is coupled to a second, opposite edge 336 of second PCB 304. Further, a fifth thermally conductive wing 340 is coupled to a first edge 342 of third PCB 306, and a sixth thermally conductive wing 344 is coupled to a second, opposite edge 346 of third PCB 306. Fifth and sixth thermally conductive wings 340 and 344 enable connections to hermetic feedthroughs, for example. In addition, a seventh thermally conductive wing 350 is coupled to a first edge 352 of fourth PCB 308, and an eighth thermally conductive wing 354 is coupled to a second, opposite edge 356 of fourth PCB 308. Thermally conductive wings 320, 324, 330, 334, 340, 344, 350, 354, may be, for example, made of copper, and may be somewhat flexible. Alternatively, thermally conductive wings 320, 324, 330, 334, 340, 344, 350, 354 may be made of any suitable material with a relatively high thermal heat transfer coefficient.

In some embodiments, thermally conductive wings are only coupled to some of PCBs 302, 304, 306, and 308. For example, in one embodiment, only third thermally conductive wing 330 and fourth thermally conductive wing 334 (both coupled to second PCB 304) are included in electronics layout 300.

Notably, heat flux (q) flows along the path with the lowest thermal resistance. Specifically, heat flux may be represented by q=(ΔT*A*λ)/L, wherein ΔT is the change in temperature, A is the cross-sectional area through which the heat flows, L is the distance the heat flows, and, is a thermal heat transfer coefficient.

Electronics layout 300 is positioned in a folded configuration, as shown in FIG. 4, when located inside the implantable battery pack. As shown in FIG. 4, in the folded configuration, first PCB 302, second PCB 304, and fourth PCB 308 are stacked vertically, with second PCB 304 positioned between first PCB 302 and fourth PCB 308. Second PCB 304 (which includes power conversion electronics and battery charging electronics) generally generates the most heat. Accordingly, second PCB 304 is centrally located within folded electronics layout 300 (and implantable battery pack). Third PCB 306 is positioned on a side of folded electronics layout 300, and is oriented generally perpendicular to first, second, and fourth PCBs 302, 304, and 308.

Without the thermally conductive wings, heat would flow to the center of the top and bottom of the implantable battery pack (i.e., above the center of the first PCB 302 and below the center of the fourth PCB 308). The heat would flow this way because the distance (L) is small and the cross-sectional area (A) is large, even though the thermal heat transfer coefficient (Q) of air is relatively small.

Accordingly, to effectively manage heat, a thermally conductive material (e.g., the thermally conductive wings) is used to increase heat flux towards the sides of electronics layout 300 (and the implantable battery pack). Further, the thermally conductive wings are connected to the components that generate relatively large amounts of heat by thermal vias (not shown). To increase heat dissipation even more, the thermal vias may be filled with copper.

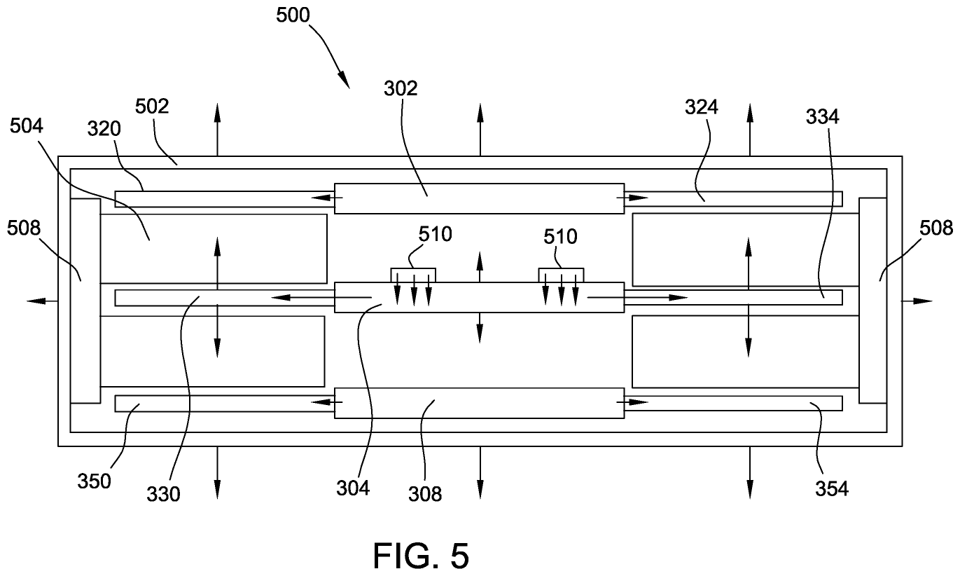

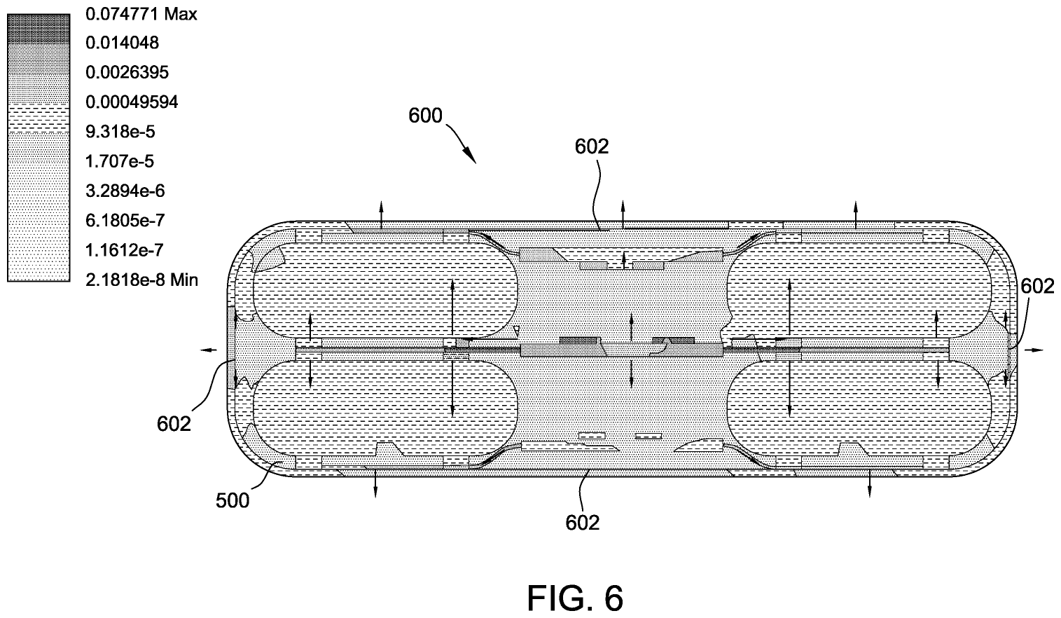

As shown in FIGS. 3A, 3B, and 4, third thermally conductive wing 330 and fourth thermally conductive wing 334 include solder connections 360 for coupling battery cells (e.g., lithium ion cells) to third and fourth thermally conductive wings 330 and 334. Battery cells are positioned between thermally conductive wings, as shown in FIGS. 5 and 6 (discussed below).

To spread heat, thermally conductive wings conduct heat to the batteries and the housing of the implantable battery pack. Further, an inner side of the housing may be laminated with graphite foils to improve heat dissipation. The graphite foils have an anisotropic heat conductivity, such that they may conduct heat approximately 800 times greater in plane than out of plane.

FIG. 5 is a schematic diagram illustrating heat flow through an implantable battery pack 500 including electronics layout 300. As shown in FIG. 5, implantable battery pack includes electronics assembly 300 positioned within an outer housing 502. Arrows in FIG. 5 represent heat flow through implantable battery pack 500.

As noted above, in implantable battery pack 500, battery cells 504 are positioned between thermally conductive wings 320, 324, 330, 334, 350, and 354. Further, plastic fixtures 508 are positioned between battery cells 504 and outer housing 502. As shown in FIG. 5, from circuitry 510 on second PCB 304, heat flows through second PCB 304, out through thermally conductive wings 330 and 334, and out through battery cells 504 and plastic fixtures 508 to outer housing 502. To further enhance heat spreading, plastic fixtures 508 may also be fabricated from a material with a desired thermal conductivity. Heat similarly flows out through thermally conductive wings 320, 324, 350, and 354 from first PCB 302 and fourth PCB 308. These heat flows result in heat being much more evenly spread to the outer housing 502, instead of heat being focused on only a few spots on outer housing 502.

FIG. 6 shows a thermal simulation 600 of the heat flow density [W/mm²] for implantable battery pack 500. As shown in thermal simulation 600, heat from circuitry 510 is dispersed and distributed relatively evenly throughout implantable battery pack. In FIG. 6, graphite foils 602 are also visible lining the inner surface of outer housing 502. As noted above, graphite foils 602 further improve heat spreading throughout implantable battery pack 500.

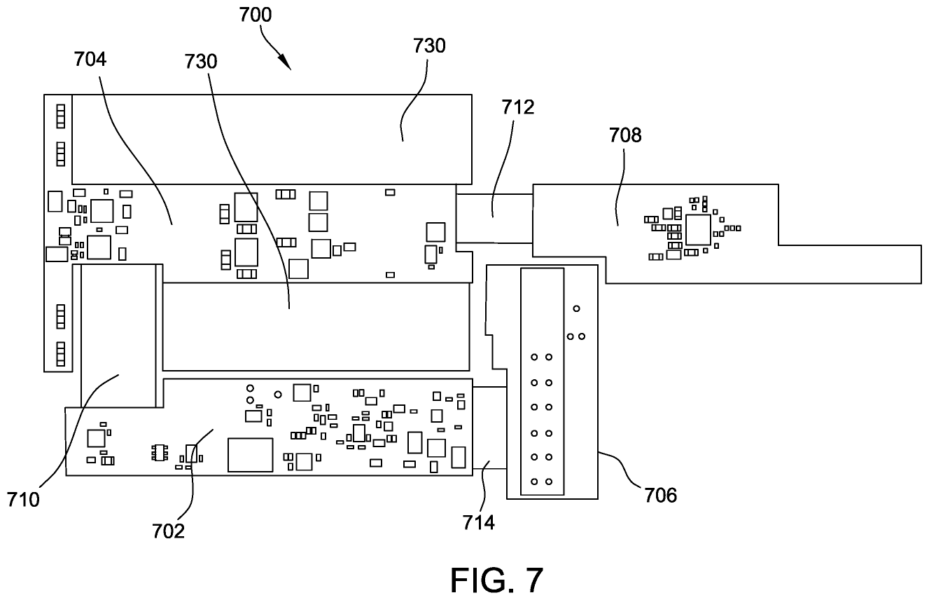

FIG. 7 is a plan view of an alternative electronics layout 700 in an unfolded configuration. Electronics layout 700 includes a first PCB 702, a second PCB 704, a third PCB 706, and a fourth PCB 708. In this embodiment, a microcontroller and digital signal processing electronics are mounted to first PCB 702, power conversion electronics and battery charging electronics are mounted to second PCB 704, and boost electronics (e.g., a DC-DC step up converter electronics) are mounted to fourth PCB 708. Alternatively, any suitable electronics may be mounted to PCBs 702, 704, 706, and 708.

Further, a first thermally conductive connector 710 extends between first PCB 702 and second PCB 704, a second thermally conductive connector 712 extends between second PCB 704 and fourth PCB 708, and a third thermally conductive connector 714 extends between first PCB 702 and third PCB 706. Conductive connectors 710, 712, and 714 may be, for example, flexible PCBs that extend between relatively rigid PCBs 702, 704, 706, and 708.

To facilitate spreading heat, in this embodiment, electronics layout 700 includes two thermally conductive wings 730 coupled to second PCB 704. In contrast to electronics layout 300 (shown in FIGS. 3A, 3B, and 4), no thermally conductive wings are coupled to the remaining PCBs 702, 706, and 708. Thermally conductive wings 730 may be, for example, made of copper, and may be somewhat flexible. Alternatively, thermally conductive wings 730 may be made of any suitable material with a relatively high thermal heat transfer coefficient.

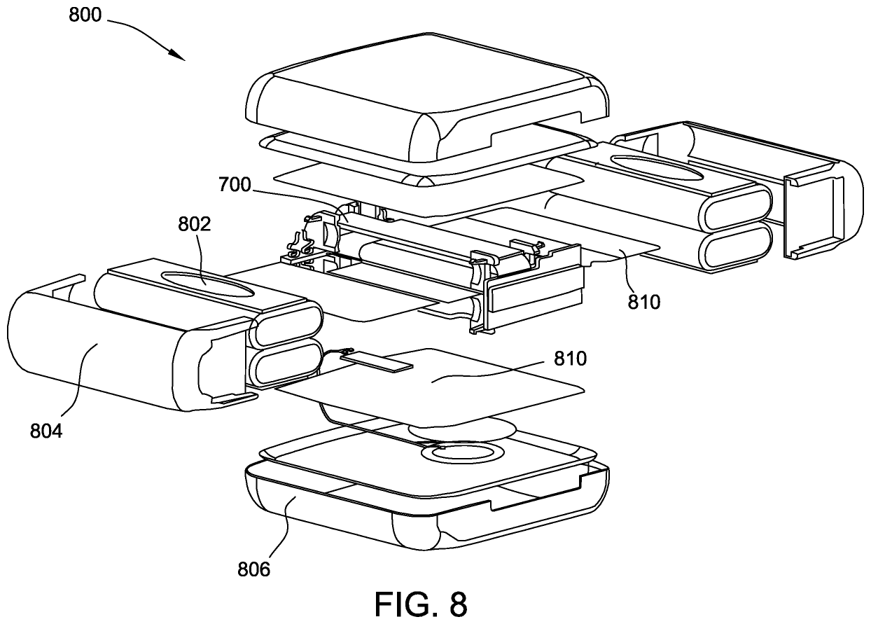

FIG. 8 is an exploded view of an alternative implantable battery pack 800 including electronics layout 700. Similar to electronics layout 300, electronics layout 700 is positioned in a folded configuration when located in implantable battery pack 800. As shown in FIG. 8, implantable battery pack 800 includes electronics layout 700, battery cells 802 (e.g., lithium ion cells), a plastic fixture 804, and an outer titanium housing 806. Further, implantable battery pack 800 also includes graphite foils 810. Although two graphite foils 810 are shown, additional graphite foils may also be included (e.g., above and below electronics layout 300).

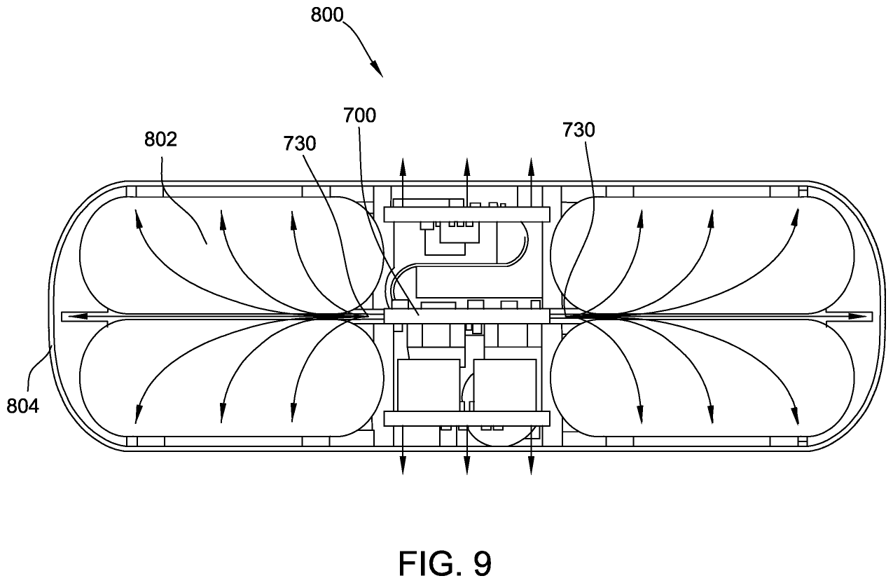

FIG. 9 is a schematic diagram illustrating heat flow through implantable battery pack 800 including electronics layout 700. Arrows in FIG. 9 represent heat flow through implantable battery pack 800. As shown in FIG. 9, although thermally conductive wings 330, 730 only extend from second PCB 704, heat still spreads out from second PCB 704 substantially evenly through battery cells 802 to outer housing 804.

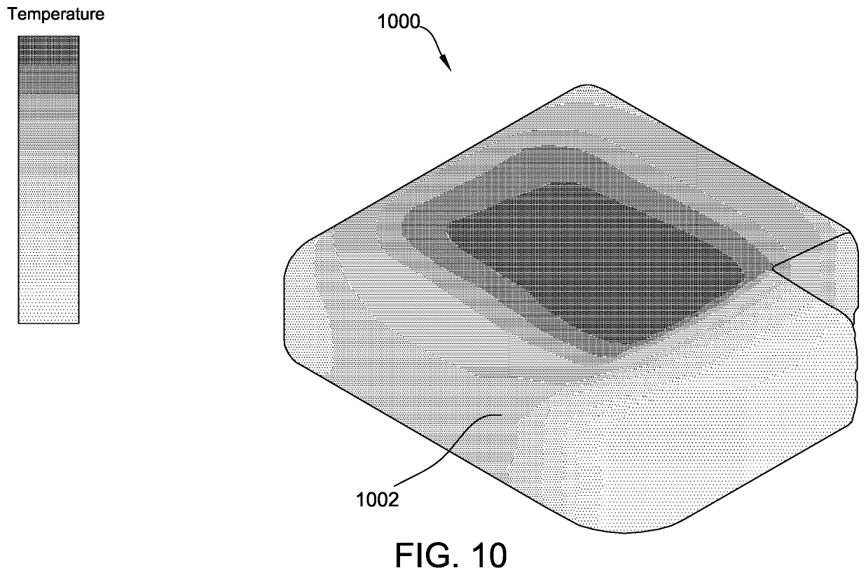

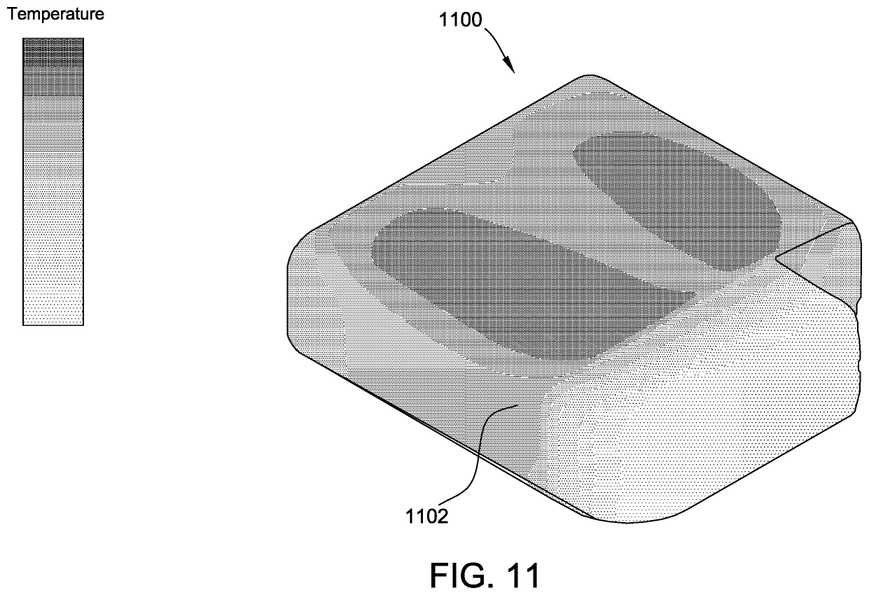

As explained above, the systems and methods described herein facilitate spreading heat much more evenly throughout an implantable battery pack. For example, FIG. 10 shows a thermal simulation 1000 for an implantable battery pack 1002 that does not implement the systems and methods described herein, while FIG. 11 shows a thermal simulation 1100 for an implantable battery pack 1102 that does implement the systems and methods described herein. As shown in FIGS. 10 and 11, the thermal energy is focused in one spot on an exterior surface of implantable battery pack 1002, while the thermal energy is more evenly spread over the exterior surface implantable battery pack 1102. Further, the exterior surface maximum temperature of implantable battery pack 1102 is 0.5° C. lower than the exterior surface maximum temperature of the implantable battery pack 1002.

The embodiments described herein are directed to systems and methods for managing heat in wireless power transfer systems. An implantable battery pack includes a housing, a plurality of battery cells positioned within the housing, and an electronics layout positioned within the housing, the electronics layout electrically coupled to the plurality of battery cells. The electronics layout includes at least one printed circuit board including electronics mounted thereon, and at least one thermally conductive wing coupled to and extending from the at least one printed circuit board, the at least one thermally conductive wing operable to spread heat generated by the electronics throughout the implantable battery pack.

Although the embodiments and examples disclosed herein have been described with reference to particular embodiments, it is to be understood that these embodiments and examples are merely illustrative of the principles and applications of the present disclosure. It is therefore to be understood that numerous modifications can be made to the illustrative embodiments and examples and that other arrangements can be devised without departing from the spirit and scope of the present disclosure as defined by the claims. Thus, it is intended that the present application cover the modifications and variations of these embodiments and their equivalents.

This written description uses examples to disclose the disclosure, including the best mode, and also to enable any person skilled in the art to practice the disclosure, including making and using any devices or systems and performing any incorporated methods. The patentable scope of the disclosure is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

What is claimed is:

1. An implantable battery pack comprising:
a housing;
a plurality of battery cells positioned within the housing; and
an electronics layout positioned within the housing, the electronics layout electrically coupled to the plurality of battery cells and comprising:
at least one printed circuit board including electronics mounted thereon; and
at least one thermally conductive wing coupled to and extending from the at least one printed circuit board, the at least one thermally conductive wing operable to spread heat generated by the electronics throughout the implantable battery pack.

2. The implantable battery pack of claim 1, wherein the plurality of battery cells comprise a plurality of lithium ion battery cells.

3. The implantable battery pack of claim 1, wherein the at least one printed circuit board comprises a first printed circuit board including power conversion electronics and battery charging electronics mounted thereon, and wherein the first printed circuit board is located proximate a center of the implantable battery pack.

4. The implantable battery pack of claim 2, wherein the at least one thermally conductive wing comprises:
a first thermally conductive wing extending from a first edge of the first printed circuit board; and
a second thermally conductive wing extending from a second, opposite edge of the first printed circuit board.

5. The implantable battery pack of claim 1, further comprising at least one graphite sheet positioned within the housing, the at least one graphite sheet configured to further spread heat generated by the electronics.

6. The implantable battery pack of claim 1, wherein the at least one thermally conductive wing is copper.

7. The implantable battery pack of claim 1, wherein the at least one thermally conductive wing comprises at least one solder connection for electrically coupling the at least one printed circuit board to the plurality of battery cells.

8. An electronics layout for use in an implantable battery pack including a housing and a plurality of battery cells, the electronics layout comprising:
at least one printed circuit board including electronics mounted thereon; and
at least one thermally conductive wing coupled to and extending from the at least one printed circuit board, the at least one thermally conductive wing operable to spread heat generated by the electronics throughout the implantable battery pack.

9. The electronics layout of claim 8, wherein the at least one printed circuit board comprises a first printed circuit board including power conversion electronics and battery charging electronics mounted thereon, and wherein the first printed circuit board is located proximate a center of the implantable battery pack.

10. The electronics layout of claim 9, wherein the at least one thermally conductive wing comprises:
a first thermally conductive wing extending from a first edge of the first printed circuit board; and
a second thermally conductive wing extending from a second, opposite edge of the first printed circuit board.

11. The electronics layout of claim 8, wherein the at least one thermally conductive wing is copper.

12. The electronics layout of claim 8, wherein the at least one thermally conductive wing comprises at least one solder connection for electrically coupling the at least one printed circuit board to the plurality of battery cells.

13. The electronics layout of claim 8, wherein the at least one printed circuit board comprises:
a first printed circuit board;
a second printed circuit board connected to the first printed circuit board by a first flexible thermally conductive connector;
a third printed circuit board connected to the second printed circuit board by a second flexible thermally conductive connector; and
a fourth printed circuit board connected to the third printed circuit board by a third flexible thermally conductive connector, wherein the electronics layout is positionable in a folded configuration in which the second printed circuit board is stacked between the first printed circuit board and the fourth printed circuit board.

14. A method of assembling an implantable battery pack, the method comprising:
positioning a plurality of battery cells within a housing; and
electrically coupling an electronics layout to the plurality of battery cells, the electronics layout positioned within the housing and including at least one printed circuit board including electronics mounted thereon, and at least one thermally conductive wing coupled to and extending from the at least one printed circuit board, the at least one thermally conductive wing operable to spread heat generated by the electronics throughout the implantable battery pack.

15. The method of claim 14, wherein the plurality of battery cells include a plurality of lithium ion battery cells.

16. The method of claim 14, wherein the at least one printed circuit board includes a first printed circuit board including power conversion electronics and battery charging electronics mounted thereon, and wherein the first printed circuit board is located proximate a center of the implantable battery pack.

17. The method of claim 16, wherein the at least one thermally conductive wing includes a first thermally conductive wing extending from a first edge of the first printed circuit board, and a second thermally conductive wing extending from a second, opposite edge of the first printed circuit board.

18. The method of claim 14, further comprising positioning at least one graphite sheet within the housing, the at least one graphite sheet configured to further spread heat generated by the electronics.

19. The method of claim 14, wherein the at least one thermally conductive wing is copper.

20. The method of claim 14, wherein the at least one thermally conductive wing includes at least one solder connection for electrically coupling the at least one printed circuit board to the plurality of battery cells.
